(19) United States
(12) Patent Application Publication
(10) Pub. No.: US 2026/0100143 A1
(43) Pub. Date: Apr. 9, 2026

(54) DEMENTIA DETECTION DEVICE, DEMENTIA DETECTION METHOD, AND PROGRAM

(71) Applicant: NEC Corporation, Tokyo (JP)

(72) Inventors: Minami SAEGUSA, Tokyo (JP); Rui MATSUO, Tokyo (JP); Hiroshi SATOU, Tokyo (JP); Suzune UNGA, Tokyo (JP); Tatsuki OGISHI, Tokyo (JP); Keisuke GOGUN, Tokyo (JP)

(73) Assignee: NEC Corporation, Tokyo (JP)

(21) Appl. No.: 19/344,776

(22) Filed: Sep. 30, 2025

(30) Foreign Application Priority Data
Oct. 9, 2024 (JP) 2024-177094

(51) Int. Cl. G09B 19/00; A61B 5/00; G16H 80/00

(52) U.S. Cl. CPC G09B 19/00; A61B 5/4088; A61B 5/742; G16H 80/00

(57) ABSTRACT

In order to provide an opportunity for the early detection of dementia, a dementia detection device communicates with a subject terminal used by a subject and a family member terminal used by each of members of a family, stores personal information regarding the subject and each member and photograph information related to the subject based on consent of the subject and each member, transmits the photograph information to the subject terminal and displaying the photograph information on the subject terminal, has a conversation with the subject using an AI model based on the personal information and the photograph information via the subject terminal, determines whether memories of the subject are unclear by analyzing content of the conversation based on the personal information and the photograph information for decision making regarding medical institution consultation, and notifies the family member terminal if the memories of the subject are unclear.

### INCORPORATION BY REFERENCE

This application is based upon and claims the benefit of priority from Japanese Patent Application 2024-177094, filed on Oct. 9, 2024, the disclosure of which is incorporated herein in its entirety by reference.

### TECHNICAL FIELD

The present disclosure relates to a technology for providing an opportunity for the early detection of dementia.

### BACKGROUND ART

In recent years, in Japan, the aging population is increasing, and the number of dementia patients is increasing. In an aging society, the problem of dementia is a serious concern, early detection has become an issue. Patent Document 1 describes an electronic device-based system for determining the presence or absence of suspected dementia from daily conversations.

Patent Document 1: Japanese Patent Application Laid-Open under No. 2023-010524

### SUMMARY

The risk of dementia tends to increase with a low amount of conversation. However, since the elderly persons are away from their family and live alone, the number of elderly persons who live alone with a low amount of conversation has been increasing year by year. Even elderly persons who live together with their family or elderly persons who live in a facility tend to have little daily conversation due to a problem that their family is busy or the number of care providers is small.

An object of the present disclosure is to provide an opportunity for the early detection of dementia.

According to an example aspect of the present invention, there is provided a dementia detection device comprising:
at least one memory configured to store instructions; and
at least one processor configured to execute the instructions to:
communicate with a subject terminal used by a subject and a family member terminal used by each of members of a family;
store personal information regarding the subject and each member and photograph information related to the subject based on consent of the subject and each member;
transmit the photograph information to the subject terminal and displaying the photograph information on the subject terminal;
have a conversation with the subject using an AI model based on the personal information and the photograph information via the subject terminal;
determine whether memories of the subject are unclear by analyzing content of the conversation based on the personal information and the photograph information; and
notify the family member terminal in a case where the memories of the subject are unclear.

According to another example aspect of the present invention, there is provided a dementia detection method executed by a dementia detection device, the dementia detection method comprising:
communicating with a subject terminal used by a subject and a family member terminal used by each of members of a family;
storing personal information regarding the subject and each member and photograph information related to the subject based on consent of the subject and each member;
transmitting the photograph information to the subject terminal and displaying the photograph information on the subject terminal;
having a conversation with the subject using an AI model based on the personal information and the photograph information via the subject terminal;
determining whether memories of the subject are unclear by analyzing content of the conversation based on the personal information and the photograph information; and
notifying the family member terminal in a case where the subject memories are unclear.

According to still another example aspect of the present invention, there is provided a recording medium recording a program, the program causing a computer to execute processing of:
communicating with a subject terminal used by a subject and a family member terminal used by each of members of a family;
storing personal information regarding the subject and each member and photograph information related to the subject based on consent of the subject and each member;
transmitting the photograph information to the subject terminal and displaying the photograph information on the subject terminal;
having a conversation with the subject using an AI model based on the personal information and the photograph information via the subject terminal;
determining whether memories of the subject are unclear by analyzing content of the conversation based on the personal information and the photograph information; and
notifying the family member terminal in a case where the subject memories are unclear.

### EFFECT

According to the present disclosure, it is possible to provide an opportunity for early detection of dementia.

### BRIEF DESCRIPTION OF THE DRAWINGS

FIG. 1 illustrates an example of a schematic configuration of a dementia detection system;

FIGS. 2A and 2B illustrate examples of hardware configurations of a server and a subject terminal;

FIG. 3 is a diagram illustrating data registered in various DBs;

FIG. 4 is a block diagram illustrating an example of a functional configuration of the server;

FIG. 5 is a diagram illustrating registration of information via a card information acquisition API;

FIGS. 6A and 6B are diagrams illustrating functions of a photograph correction unit;

FIG. 7 is an example of evaluation data;

FIG. 8 is a diagram illustrating a flow until disclosed information is provided to a medical institution;

FIG. 9 is a flowchart of memory determination processing;

FIG. 10 is an example of a photograph subjected to mosaic processing;

FIG. 11 is a block diagram illustrating a functional configuration of a dementia detection device according to a second example embodiment; and

FIG. 12 is a flowchart of processing by a dementia detection device according to the second example embodiment.

### EXAMPLE EMBODIMENTS

Hereinafter, example embodiments of the present disclosure will be described with reference to the drawings.

### First Example Embodiment

### (Overall Configuration)

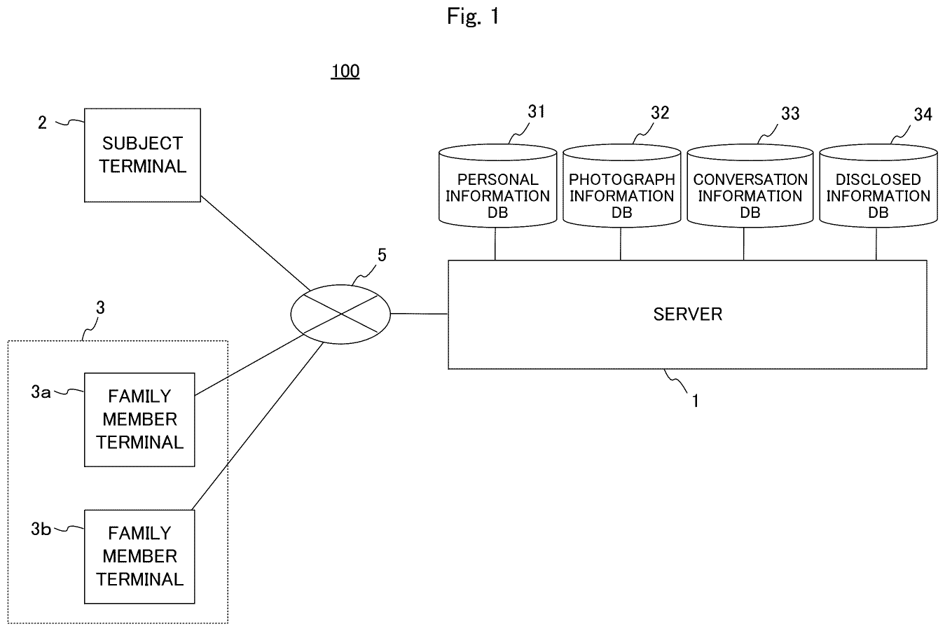

FIG. 1 is an example of a schematic configuration of a dementia detection system 100 to which a dementia detection device of the present disclosure is applied. The dementia detection system 100 is a system that conducts a conversation with a subject using an AI model, and notifies a family member of the subject based on an analysis result, thereby providing an opportunity for early detection of dementia.

Specifically, the AI model is trained on past photographs and videos related to the subject in advance, and then provides a notification of suspected dementia and information regarding a situation based on the conversation history while having a conversation about past memories with the subject. In other words, the dementia detection system 100 provides the subject and his/her family members with a service for preventing or detecting dementia early by conversation between the AI model and the subject.

In the dementia detection system 100 of FIG. 1, a server 1, a subject terminal 2, a family member terminal 3a, and a family member terminal 3b are communicably connected via a network 5 such as the Internet. The family member terminals 3a and 3b are also collectively referred to as family member terminal 3.

In the present example embodiment, as an example, the subject is an elderly person who does not have enough daily conversation. It is assumed that the family includes two members: an eldest son and second son of the subject. The subject uses the subject terminal 2, the eldest son uses the family member terminal 3a, and the second son uses the family member terminal 3b. The members of the family are not limited to two persons who are biologically related to the subject, and include at least one arbitrary member who has a strong connection with the subject such as a family member and a relative.

The subject terminal 2 is a smartphone, a tablet, a PC, or the like used by the subject, and receives audio data for the AI model to have a conversation with the subject from the server 1 or transmits audio data for the subject to have a conversation with the AI model to the server 1. The subject terminal 2 is an example of the subject terminal of the present disclosure.

The family member terminal 3 is a smartphone, a tablet, a PC, or the like used by each member, receives a notification indicating that memories of the subject are unclear from the server 1, or transmits and registers memorable photographs with the subject to the server 1. The family member terminal 3 is an example of the family member terminal of the present disclosure.

The server 1 is an information processing device that performs processing, storage, and transmission/reception of various data, and includes an AI model. The server 1 is connected to a personal information database (hereinafter, “database” is referred to as “DB”) 31, a photograph information DB 32, a conversation information DB 33, and a disclosed information DB 34 to be described later. The AI model is a large language model (LLM) capable of understanding multimodal information, which has a conversation with the subject via the subject terminal 2. Specifically, the AI model exchanges information with the personal information DB 31, the photograph information DB 32, the conversation information DB 33, and the disclosed information DB 34, thereby creating content of the conversation with the subject and selecting a photograph related to the conversation.

The server 1 stores personal information regarding the subject and his/her family members and photograph information in various DBs, transmits audio data for the AI model to have a conversation with the subject and a photograph related to the conversation to the subject terminal 2, and receives audio data for the subject to have a conversation with the AI model and personal information regarding the subject from the subject terminal 2. The server 1 analyzes the conversation between the AI model and the subject, and determines whether memories of the subject are unclear. In a case where the subject memories are unclear, the server 1 notifies the family member terminal 3 that the subject is suspected of having dementia and thus a visit to a medical institution is recommended.

The server 1 may be a virtual server in a cloud environment. The server 1 is an example of the dementia detection device of the present disclosure.

### (Hardware Configuration)

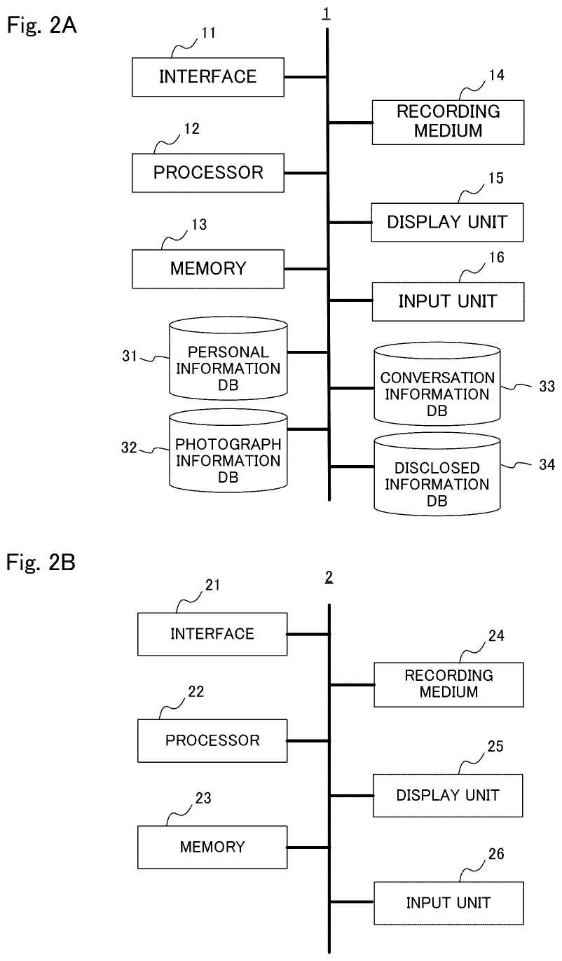

FIG. 2A is a block diagram illustrating an example of a hardware configuration of the server 1. As illustrated in FIG. 2A, the server 1 includes an interface 11, a processor 12, a memory 13, a recording medium 14, a display unit 15, and an input unit 16. These components, the personal information DB 31, the photograph information DB 32, the conversation information DB 33, and the disclosed information DB 34 are mutually connected via a bus.

The interface 11 exchanges data with the subject terminal 2 or the family member terminal 3. The interface 11 is used to receive audio data and personal information for the subject to have a conversation with the AI model from the subject terminal 2, and transmit audio data and a photograph related to conversation for the AI model to have a conversation with the subject to the subject terminal 2.

The interface 11 receives personal information and photograph information from the family member terminal 3, and transmits a notification indicating that the subject memories are unclear to the family member terminal 3.

The processor 12 is a computer such as a central processing unit (CPU), and controls the entire server 1 by executing a program prepared in advance. As the processor 12, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, a combination of these, or the like can be used.

The memory 13 includes a read only memory (ROM) and a random access memory (RAM). The memory 13 stores a program executed by the processor 12. The memory 13 is also used as a work memory during execution of various types of processing by the processor 12.

The recording medium 14 is a non-volatile non-transitory recording medium such as a disk-shaped recording medium or a semiconductor memory, and is attachable to and detachable from the server 1. The recording medium 14 records various programs executed by the processor 12. When the server 1 executes memory determination processing, the program recorded in the recording medium 14 is loaded into the memory 13 and executed by the processor 12.

The display unit 15 displays a predetermined image by, for example, a liquid crystal display (LCD). The input unit 16 is a keyboard, a mouse, a touch panel, or the like, and is used by an operator who manages the server 1.

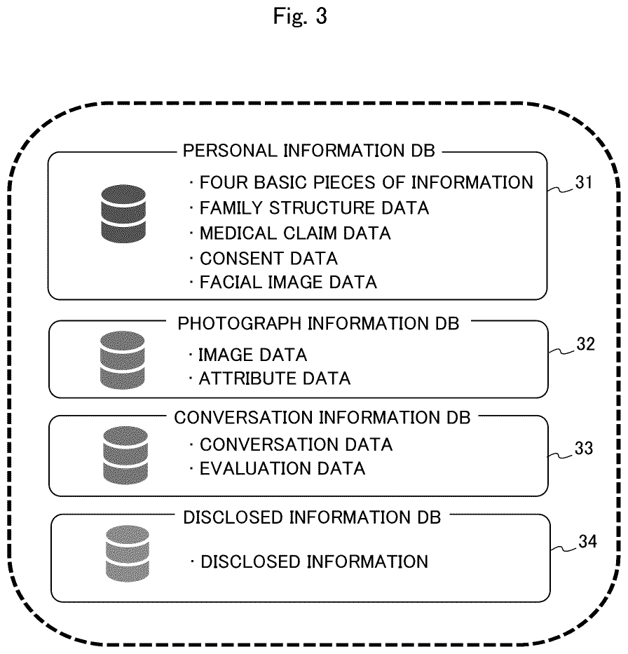

FIG. 3 is a diagram illustrating data registered in the various DBs. As illustrated in FIG. 3, the personal information DB 31 stores four basic pieces of information, family structure data, medical claim data of the subject, consent data, and facial image data as personal information. The four basic pieces of information include a name, an address, a date of birth, and a gender of the subject and each member of a family. The family structure data is data indicating a family structure, and is, for example, kinship indicating a relationship between each member and the subject, such as the eldest son and the second son. The medical claim data is a medical fee statement submitted by a medical institution to an insurer, and it is possible to acquire medication information regarding a currently treated disease or medication from the medical claim data of the subject. The consent data is data indicating consent to the services provided by the dementia detection system 100, such as use of facial image data of the subject and family members, acquisition of the medical claim data of the subject, and reception of notification that memories of the subject are unclear. The facial image data is current facial image data of the subject and each member.

The consent data may be one piece of consent data for the entire service, or may be consent data for each item necessary for providing a service including use of facial images or acquisition of medical claim data. In the present example embodiment, for convenience, an example will be described in which the subject and each member of the family give consent to the entire service.

In the photograph information DB 32, as illustrated in FIG. 3, image data and attribute data of a photograph related to the subject are stored as photograph information. The photograph related to the subject is a memorable photograph or the like in which a subject or a family member appears. The attribute data is a photograph capture time, a person appearing in the photograph, a photograph capture location, an episode regarding the photograph, and the like.

As illustrated in FIG. 3, the conversation data and the evaluation data are stored in the conversation information DB 33 as conversation information. Specifically, the server 1 stores audio data presented by the AI model to the subject terminal 2 and audio data acquired from the subject terminal 2 in the conversation information DB 33 as conversation data including the date and time of the conversation. The server 1 creates evaluation data obtained by evaluating memories of the subject and stores the evaluation data in the conversation information DB 33.

As illustrated in FIG. 3, the disclosed information DB 34 stores information disclosed to a medical institution. Specifically, in a case where the subject visits a medical institution due to suspected dementia, the server 1 stores the disclosed information disclosed to the medical institution in the disclosed information DB 34. The disclosed information is, for example, medical claim data of the subject, evaluation data, conversation data when the memories of the subject are unclear, or the like.

FIG. 2B is a block diagram illustrating an example of a hardware configuration of the subject terminal 2. As illustrated in FIG. 2B, the subject terminal 2 includes an interface 21, a processor 22, a memory 23, a recording medium 24, a display unit 25, and an input unit 26.

The interface 21 exchanges data with the server 1 via the network 5. The interface 21 is used to transmit, to the server 1, audio data and personal information for the subject to have a conversation with the AI model, and receive audio data and a photograph related to conversation for the AI model to have a conversation with the subject from the server 1.

The processor 22 is a computer such as a CPU, and controls the entire subject terminal by executing a program prepared in advance. As the processor 22, a CPU, a GPU, a DSP, an MPU, an FPU, a PPU, a TPU, a quantum processor, a microcontroller, or a combination of these can be used.

The memory 23 includes a ROM and a RAM. The memory 23 stores a program executed by the processor 22. The memory 23 is also used as a work memory during execution of various types of processing by the processor 22.

The recording medium 24 is a non-volatile non-transitory recording medium such as a disk-shaped recording medium or a semiconductor memory, and is attachable to and detachable from the subject terminal 2. The recording medium 24 records various programs executed by the processor 22. The display unit 25 displays a predetermined image by, for example, an LCD. The input unit 26 is a touch panel or the like, and is used when a user performs a predetermined operation.

Since the hardware configuration of the family member terminal 3 is similar to that of the subject terminal 2, the description thereof will be omitted for convenience.

### (Functional Configuration)

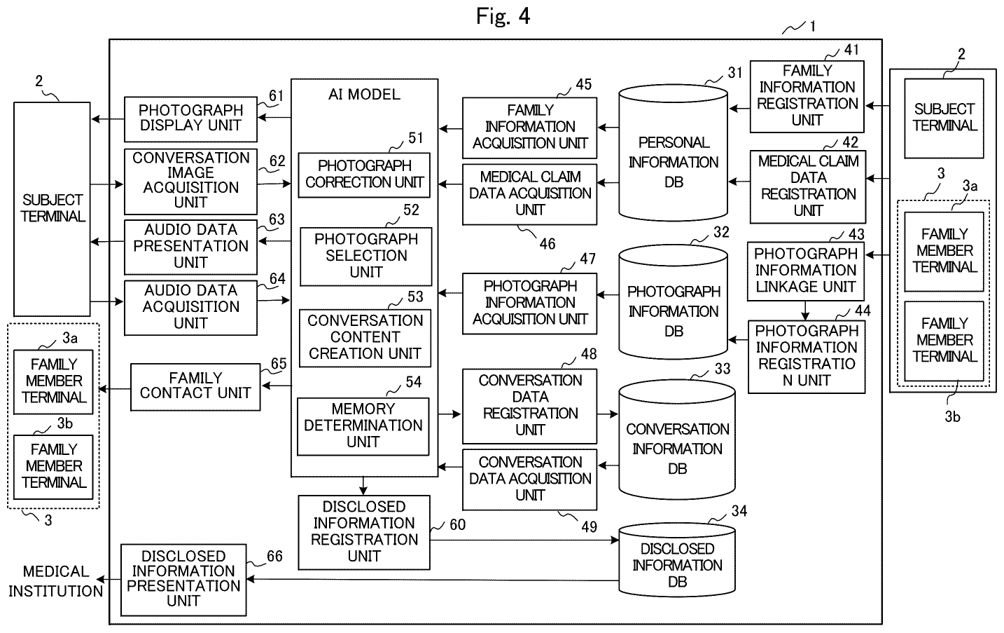

FIG. 4 is a block diagram illustrating an example of a functional configuration of the server 1. The server 1 includes the personal information DB 31, the photograph information DB 32, a conversation information DB 33, and the disclosed information DB 34. The server 1 functionally includes a family information registration unit 41, a medical claim data registration unit 42, a photograph information linkage unit 43, a photograph information registration unit 44, a family information acquisition unit 45, a medical claim data acquisition unit 46, a photograph information acquisition unit 47, a conversation data registration unit 48, a conversation data acquisition unit 49, an AI model, a disclosed information registration unit 60, a photograph display unit 61, a conversation image acquisition unit 62, an audio data presentation unit 63, an audio data acquisition unit 64, a family contact unit 65, and a disclosed information presentation unit 66. The AI model functions as a photograph correction unit 51, a photograph selection unit 52, a conversation content creation unit 53, and a memory determination unit 54.

The family information registration unit 41, the medical claim data registration unit 42, the photograph information linkage unit 43, the photograph information registration unit 44, the family information acquisition unit 45, the medical claim data acquisition unit 46, the photograph information acquisition unit 47, the conversation data registration unit 48, the conversation data acquisition unit 49, the AI model, the disclosed information registration unit 60, the photograph display unit 61, the conversation image acquisition unit 62, the audio data presentation unit 63, the audio data acquisition unit 64, the family contact unit 65, and the disclosed information presentation unit 66 are implemented by the processor 12 executing a program.

When starting the service provided by the dementia detection system 100, the subject and each member of the family install a predetermined application on the subject terminal 2 or the family member terminal 3. In a case where the personal information or the photograph information is registered, the subject terminal 2 or the family member terminal 3 activates the application through a predetermined operation and selects a registration mode for registering various types of information.

The family information registration unit 41 registers the four basic pieces of information and facial image data of the subject in the personal information DB 31, which are obtained from the My Number Card of the subject, using a card information acquisition application programming interface (API) via the subject terminal 2. The family information registration unit 41 acquires consent data of the subject and registers the consent data in the personal information DB 31.

The family information registration unit 41 registers the four basic pieces of information and facial image data of each member in the personal information DB 31, which are obtained from the My Number Card of each member of the family, using the card information acquisition API via the family member terminal 3. The family information registration unit 41 acquires consent data and family structure data of each member and registers the consent data and the family structure data in the personal information DB 31.

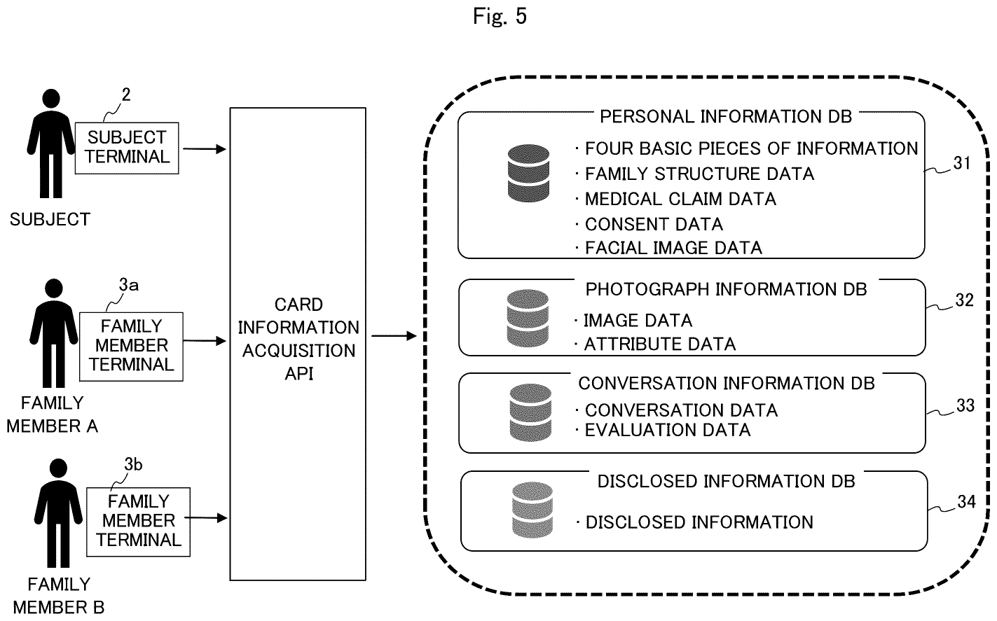

FIG. 5 is a diagram illustrating registration of information via the card information acquisition API. As illustrated in FIG. 5, the subject reads the My Number Card after giving consent to the service provided by the dementia detection system 100 through a predetermined operation using the subject terminal 2. The server 1 acquires consent data, and four basic pieces of information and facial image data of the subject held in the My Number Card from the subject terminal 2 via the card information acquisition API, and registers the consent data, and the four basic pieces of information and facial image data in the personal information DB 31.

Each member of the family reads the My Number Card after giving consent to the service provided by the dementia detection system 100 through a predetermined operation using the family member terminal 3 used by each member. At this time, each member inputs a kinship indicating a relationship with the subject as family structure data. The server 1 acquires consent data and family structure data of each member, and four basic pieces of information and facial image data held in the My Number Card from the family member terminal 3 used by each member via the card information acquisition API, and registers the acquired data and information in the personal information DB 31.

For convenience of description, the subject and each member read their own My Number Card using the terminal used by themselves, but the present disclosure is not limited thereto. As long as the My Number Card can be read and the information can be registered in various DBs via the card information acquisition API, the terminal devices used by the subject and each member are arbitrary.

The medical claim data registration unit 42 registers the medical claim data obtained from the My Number Card of the subject in the personal information DB 31 via the card information acquisition API using the subject terminal 2. The medical claim data registration unit 42 may be linked with an API for acquiring information regarding medicine, automatically acquire the medication information of the subject as needed, and store the medication information in the personal information DB 31.

The photograph information linkage unit 43 is linked with a photograph folder of the subject terminal 2 or the family member terminal 3 to acquire image data of a photograph related to the subject and attribute data including a photograph capture time and a photograph capture location. The photograph information linkage unit 43 is not limited to linkage with the photograph folder, and may acquire the photograph information by linking with a social networking service (SNS) used by the subject or the family members. The photograph information linkage unit 43 may acquire memories related to a photograph input by the subject or each family member from the subject terminal 2 or the family member terminal 3 as attribute data.

The photograph information registration unit 44 registers the image data and attribute data of the photograph as photograph information in the photograph information DB 32.

The family information acquisition unit 45 acquires four basic pieces of information, family structure data, consent data, and facial image data of the subject and each member from the personal information DB 31, and provides the acquired information and data to the AI model. The AI model is trained on the acquired four basic pieces of information, family structure data, consent data, and facial image data of the subject and each member.

The medical claim data acquisition unit 46 acquires medical claim data and medication information of the subject from the personal information DB 31 and provides the medical claim data and the medication information to the AI model.

The AI model is trained on the acquired medical claim data and medication information.

The photograph information acquisition unit 47 acquires photograph information from the photograph information DB 32 and provides the photograph information to the AI model. The AI model is trained on the acquired photograph information.

The AI model has a conversation with the subject via the subject terminal 2. The content of the conversation is mainly memorable stories related to photographs displayed on the subject terminal 2. The conversation data registration unit 48 registers the conversation content between the AI model and the subject as conversation data in the conversation information DB 33. Specifically, in order for the AI model to have a conversation with the subject, the conversation data registration unit 48 acquires audio data presented to the subject terminal 2 and audio data acquired from the subject terminal 2 as conversation data including the date and time of the conversation. The conversation data registration unit 48 registers the conversation data in the conversation information DB 33 as needed.

The conversation data acquisition unit 49 acquires the conversation data from the conversation information DB 33 and provides the conversation data to the AI model. The AI model is trained on the acquired conversation data.

The AI model functions as a photograph correction unit 51, a photograph selection unit 52, a conversation content creation unit 53, and a memory determination unit 54.

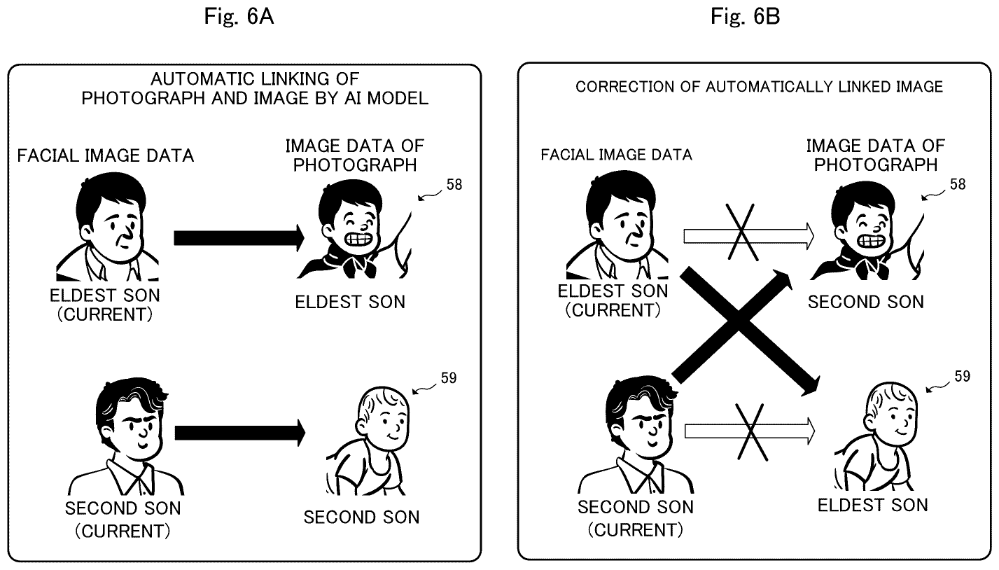

The photograph correction unit 51 links the photograph with a person shown in the photograph based on the facial image data of the subject and each member registered in the personal information DB 31, and the image data of the photograph registered in the photograph information DB 32. FIGS. 6A and 6B are diagrams illustrating functions of the photograph correction unit 51. As illustrated in FIG. 6A, for example, the photograph correction unit 51 identifies a person 58 shown in the photograph as the eldest son and a person 59 as the second son based on the current facial image data of the eldest son and second son and the image data of the photograph, and links the persons with the photograph. The photograph correction unit 51 may store link data obtained by linking the photograph with the persons shown in the photograph in the photograph information DB 32, or may classify the photograph registered in the photograph information DB 32 based on the link data.

Next, the photograph correction unit 51 displays the photograph and the persons linked with the photograph on the subject terminal 2 and the family member terminal 3 based on the link data. In a case where there is an error in the link, the subject and each member correct the link to be correct through a predetermined operation using the subject terminal 2 and the family member terminal 3. For example, as illustrated in FIG. 6B, in a case where the person 58 actually shown in the photograph is the second son and the person 59 is the eldest son, the subject and each member perform an input to correct the link error through a predetermined operation using the subject terminal 2 and the family member terminal 3. The photograph correction unit 51 updates the link data based on the input data, and the AI model is re-trained on the photograph information based on the updated link data.

The photograph correction unit 51 displays the photograph and the persons linked with the photograph based on the link data, but this is not limited thereto, and the photograph capture time, the photograph capture location, and the like of the photograph may be displayed together on the subject terminal 2 or the family member terminal 3 based on the attribute data. In this case, when there are errors in the photograph capture time and the photograph capture location, the subject and each member perform an input to correct the photograph capture time and the photograph capture location through a predetermined operation using the subject terminal 2 and the family member terminal 3. The photograph correction unit 51 updates the attribute data based on the input data, and the AI model is re-trained on the updated attribute data.

The photograph selection unit 52 selects a photograph to be transmitted to and displayed on the subject terminal 2. As a first pattern, the photograph selection unit 52 selects a photograph corresponding to the conversation content by comparing the conversation content with the subject with the personal information and the photograph information. Specifically, the photograph selection unit 52 identifies the year, the child's age at the time, and the event from the conversation content, and selects the corresponding photograph.

As a second pattern, the photograph selection unit 52 randomly selects a photograph from among the photographs registered in the photograph information DB 32 in order to start a conversation from the AI model.

A third pattern is a case where the subject is having a conversation with a predetermined member, and the photograph selection unit 52 acquires a photograph of the member with whom the subject is having a conversation as conversation image data, and identifies the member with whom the subject is having a conversation based on the conversation image data and the facial image data. Next, the photograph selection unit 52 selects a photograph in which a member having a conversation with the subject is shown.

The conversation content creation unit 53 creates content in which the AI model has a conversation with the subject via the subject terminal 2 based on the information registered in the various DBs.

The first pattern involves creating conversation content in a case of corresponding to a conversation from the subject to the AI model. In other words, it involves creating new conversation content corresponding to the conversation content with the subject or a flow. In this case, the conversation content creation unit 53 creates new conversation content based on the conversation content addressed to the AI model, the past conversation content, the photograph corresponding to the conversation content, and the like. Specifically, the AI model is trained on conversation data corresponding to the conversation content, attribute data or link data of a photograph corresponding to the conversation content, past conversation data related to the photograph, personal information of a member shown in the photograph, and the like, and creates an appropriate conversation content.

The second pattern involves creating conversation content in a case where the AI model starts a conversation. In this case, the conversation content creation unit 53 creates conversation content based on the randomly selected photograph, the past conversation content related to the photograph, and the like. Specifically, the AI model is trained on attribute data or link data of the selected photograph, the past conversation data related to the photograph, the personal information of a member shown in the photograph, and the like, and creates appropriate conversation content.

The third pattern involves creating conversation content in a case where the subject is having a conversation with a predetermined member. In this case, the conversation content creation unit 53 creates conversation content based on the photograph in which the member with whom the subject is having a conversation is shown, the past conversation content related to the photograph, and the like. Specifically, the AI model is trained on attribute data or link data of the photograph in which the member with whom the subject is having a conversation is shown, the past conversation data related to the photograph, the personal information of the member having a conversation with the subject, and the like, and creates an appropriate conversation content.

The memory determination unit 54 determines whether memories of the subject are unclear by analyzing the most recent conversation content related to the photograph based on the personal information, the photograph information, and the conversation data. Specifically, the AI model determines whether the memories of the subject are unclear by comparing the most recent conversation data related to the photograph with the attribute data or link data of the photograph.

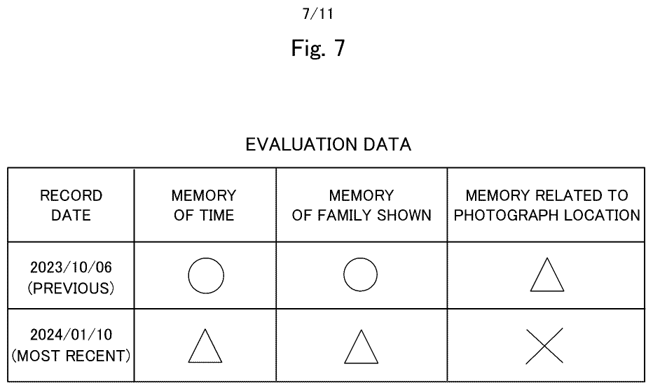

In a case where the memories of the subject are unclear, the memory determination unit 54 creates evaluation data obtained by evaluating the memories of the subject in the most recent and previous cases based on the most recent conversation data related to the photograph and the previous conversation data related to the photograph. FIG. 7 is an example of the evaluation data. Specifically, the AI model creates the evaluation data as illustrated in FIG. 7 by evaluating three types of memories: memories regarding the time of the photograph, memories regarding a family shown in the photograph, and memories regarding the photograph capture location. The created evaluation data is stored in the conversation information DB 33. In the evaluation data, the memories of the subject are represented by evaluation indicators of circles, triangles, and crosses. A circle represents “clear memory”, a triangle represents “ambiguous memory”, and a cross represents “unclear memory”. According to the evaluation data as illustrated in FIG. 7, it is possible for the subject to easily grasp the type of unclear memories and the time when the memories become unclear. The evaluation data illustrated in FIG. 7 is an example, and the type of memories and the evaluation indicator can be arbitrarily set.

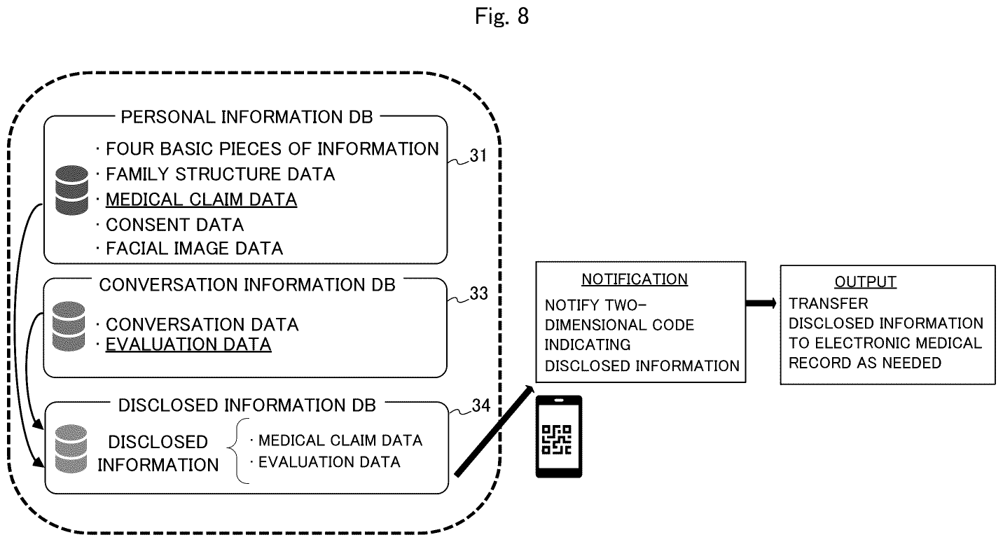

The disclosed information registration unit 60 registers information disclosed to a medical institution the subject visits in the disclosed information DB 34. FIG. 8 is a diagram illustrating a flow until the disclosed information is provided to the medical institution. As illustrated in FIG. 8, the disclosed information registration unit 60 registers, in the disclosed information DB 34, the medical claim data and medication information registered in the personal information DB 31 and the evaluation data registered in the conversation information DB 33 as the disclosed information. The disclosed information registration unit 60 may register, in the disclosed information DB 34, conversation data related to the type of memories evaluated to be unclear in the evaluation data, information regarding the photograph capture time or photograph capture location, and the like as supplementary information regarding the evaluation data.

The photograph display unit 61 displays the photograph by transmitting the image data of the photograph selected by the photograph selection unit 52 to the subject terminal 2.

In a case where the subject is having a conversation with a predetermined member, the conversation image acquisition unit 62 acquires a photograph of the member having a conversation with the subject as conversation image data. The conversation image data is, for example, image data of a photograph captured by a camera or the like of the subject terminal 2 or the family member terminal 3, and is provided to the AI model.

The audio data presentation unit 63 transmits and presents audio data for the AI model to have a conversation with the subject to the subject terminal 2.

Specifically, the audio data presentation unit 63 transmits and presents the conversation content created by the conversation content creation unit 53 as audio data to the subject terminal 2.

The audio data acquisition unit 64 acquires audio data for the subject to have a conversation with the AI model from the subject terminal 2.

In a case where the memory determination unit 54 determines that the memories of the subject are unclear, the family contact unit 65 notifies the family member terminal 3 that it is recommended to visit a medical institution due to the suspected dementia. As illustrated in FIG. 8, the family contact unit 65 outputs the disclosed information to be disclosed to the medical institution for the subject in two-dimensional code formats including a QR code (registered trademark) and notifies the family member terminal 3 of the information. The family contact unit 65 may make a notification by using an application installed when starting the service provided by the dementia detection system 100, or may register a mail address of the family member terminal 3 as a notification destination in advance as personal information and make a notification by using a mail. By making the notification, it is possible to urge the family member to confirm the state of the subject suspected of dementia or to visit the medical institution.

The disclosed information presentation unit 66 presents the disclosed information in a case where there is a request from the medical institution. As illustrated in FIG. 8, the family member who has received the notification visits a predetermined medical institution and notifies that the subject is suspected of dementia. At this time, as illustrated in FIG. 8, the family member presents the two-dimensional code transmitted to the family member terminal 3 at the medical institution. When the medical institution reads the two-dimensional code through a predetermined operation, the disclosed information presentation unit 66 transfers the disclosed information to an electronic medical record or the like as needed.

In the above-described configuration, the photograph correction unit 51, the photograph selection unit 52, the memory determination unit 54, the photograph display unit 61, and the family contact unit 65 of the server 1 are examples of a photograph correction means, a photograph selection means, a memory determination means, a photograph display means, and a notification means of the present disclosure, respectively. The conversation content creation unit 53, the audio data presentation unit 63, and the audio data acquisition unit 64 are examples of conversation means of the present disclosure. The personal information DB 31 and the photograph information DB 32 are examples of storage means of the present disclosure.

### (Memory Determination Processing)

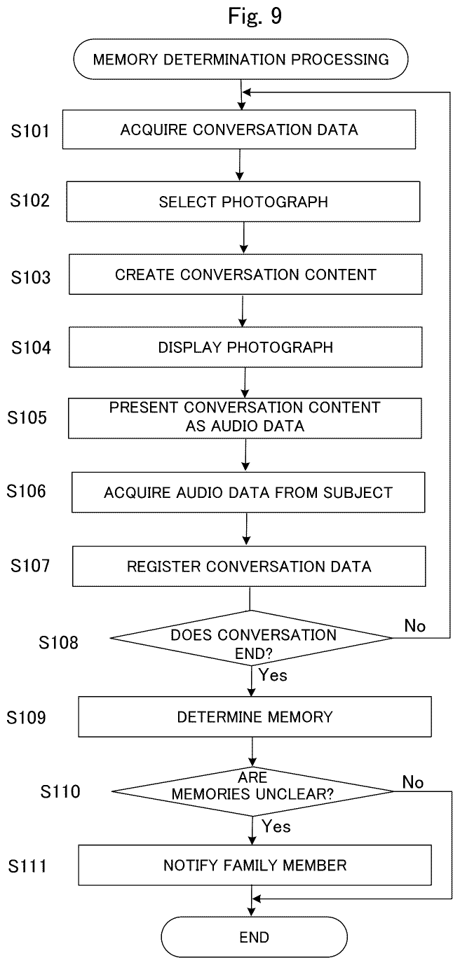

Next, memory determination processing by the server 1 will be described. FIG. 9 is a flowchart of the memory determination processing by the server 1. This processing is implemented by the processor 12 illustrated in FIG. 2A executing a program prepared in advance.

First, the server 1 acquires most recent conversation data between the AI model and the subject from the conversation information DB 33 (step S101). Next, the server 1 selects a photograph based on the acquired conversation data and information registered in various DBs using the AI model (step S102). Next, the server 1 creates new conversation content based on the acquired conversation data and the information registered in various DBs using the AI model (step S103).

The server 1 displays the photograph by transmitting the image data of the selected photograph to the subject terminal 2 (step S104). The server 1 transmits and presents the created new conversation content as audio data to the subject terminal 2 (step S105). Next, the server 1 acquires audio data for the subject to have a conversation with the AI model from the subject terminal 2 (step S106). Next, the server 1 registers the new conversation content between the AI model and the subject as conversation data in the conversation information DB 33 (step S107).

The server 1 determines whether the conversation with the subject ends (step S108). Specifically, in a case where new audio data is not acquired from the subject terminal 2, the server 1 determines that the conversation with the subject ends. In a case where it is determined that the conversation with the subject does not end (step S108; No), the server 1 returns to the processing of step S101 and continues the conversation with the subject via the subject terminal 2. On the other hand, in a case where it is determined that the conversation with the subject ends (step S108; Yes), the server 1 determines whether the memories of the subject are unclear by analyzing the most recent conversation content related to the photograph based on the information registered in the various DBs (step S109).

In a case where it is determined that the memories of the subject are not unclear (step S110; No), the server 1 ends the memory determination processing. On the other hand, in a case where it is determined that the memories of the subject are unclear (step S110; Yes), the server 1 notifies the family member terminal 3 that the subject is suspected of having dementia and thus a visit to a medical institution is recommended (step S111). At this time, the server 1 creates evaluation data and transmits, to the family member terminal 3, the two-dimensional code indicating the disclosed information including the medical claim data and the evaluation data. Then, the memory determination processing ends.

In the present disclosure, the subject terminal 2 is assumed to be a smartphone or a tablet, but is not limited thereto, and may be a robot equipped with the function of the server 1. In this case, the robot may project the photograph on a wall, a screen, or the like, or may be connected to the smartphone, a television, or the like to display the photograph. By devising the design of the robot, it is possible to motivate the subject to have a conversation as compared with the smartphone or the like.

The photograph of the present disclosure is not limited to a film photograph, and includes a digital photograph. The dementia detection system 100 of the present disclosure is not limited to a still image photograph, and a video can also be applied.

The AI model included in the server 1 creates conversation content assuming that the subject has dementia when it is determined that the subject has taken the dementia medication based on the medication information registered in the personal information DB 31. Thus, it is possible to have a conversation in consideration of the characteristics of a dementia patient different from a healthy person.

In such a dementia detection system 100, the server 1 can frequently have a conversation about the memories with a subject with a high risk of dementia such as an elderly person, by using the AI model. Receiving empathy for their memorable stories has a therapeutic effect on the subject. The server 1 can increase the amount of conversation of the subject and can prevent the dementia. The server 1 can determine whether the memories of the subject are unclear by analyzing the conversation content, and can notify the family member that the family member is recommended to visit a medical institution with the subject when the subject is suspected of dementia. Thus, it is possible to provide an opportunity for the early detection of dementia.

### First Modification Example

Based on the medical claim data registered in the personal information DB 31, the server 1 may have the AI model make inquiries of the subject related to the medication status after visiting the hospital, the physical condition after the medication, and the like. In this case, the server 1 registers the content of the inquiries as conversation data in the conversation information DB 33 and records a log. When the subject visits the hospital next time, the server 1 provides a log to the hospital using the two-dimensional code or the like, and thus the doctor and the pharmacist can easily grasp the medication status and the physical condition at home.

### Second Modification Example

The service provided by the dementia detection system 100 may be provided, for example, as an option of another AI communication service for the elderly person.

### Third Modification Example

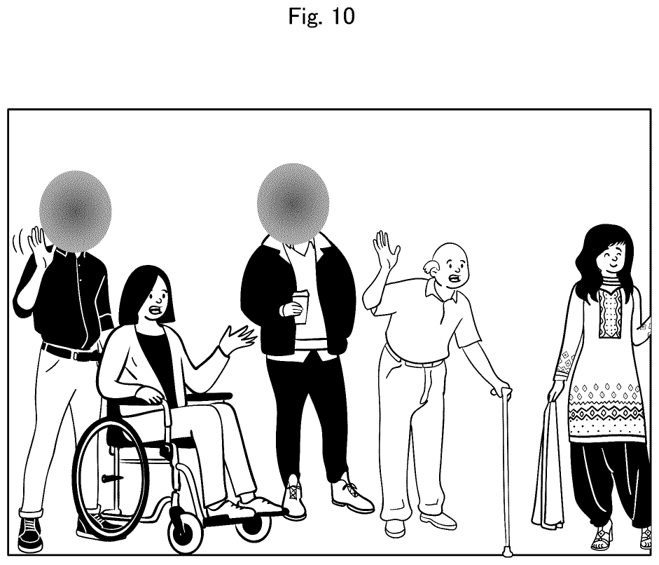

In a case where the photograph is displayed on the subject terminal 2, the server 1 may blur the face of a person other than those of the member and subject who consent to the use of facial images by mosaic processing. FIG. 10 is an example of a photograph subjected to mosaic processing. In this manner, the server 1 can protect privacy by performing mosaic processing on the faces of members who do not consent to the use of the facial image or the faces of other persons shown in the photograph.

### Second Example Embodiment

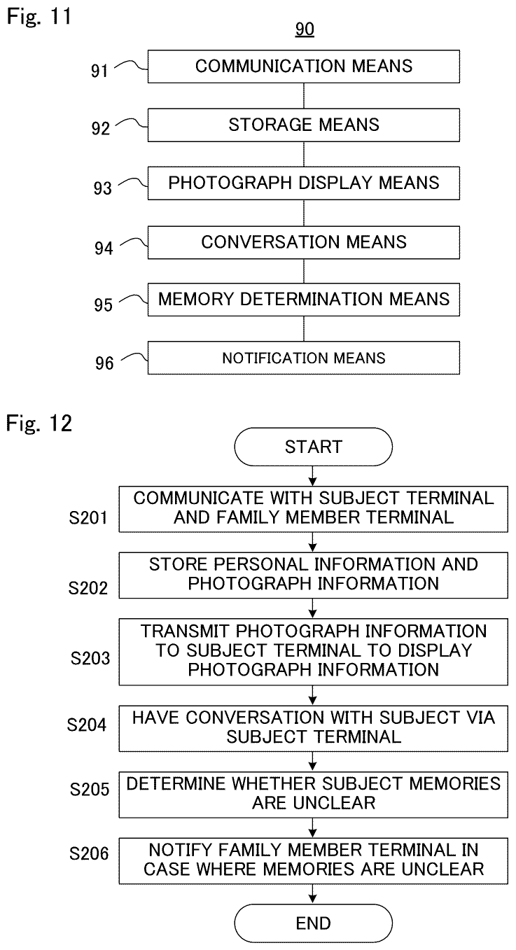

FIG. 11 is a block diagram illustrating a functional configuration of a dementia detection device according to a second example embodiment. A dementia detection device 90 includes a communication means 91, a storage means 92, a photograph display means 93, a conversation means 94, a memory determination means 95, and a notification means 96.

FIG. 12 is a flowchart of processing by the dementia detection device 90. The communication means 91 communicates with the subject terminal 2 used by the subject and the family member terminal 3 used by each member of the family (step S201). The storage means 92 stores personal information regarding a subject and each member and photograph information related to the subject based on the consent of the subject and each member (step S202). The photograph display means 93 transmits the photograph information to the subject terminal 2 to display the photograph information (step S203). The conversation means 94 has a conversation with the subject using the AI model based on the personal information and the photograph information via the subject terminal 2 (step S204). The memory determination means 95 determines whether the memories of the subject are unclear by analyzing the conversation content based on the personal information and the photograph information (step S205). In a case where the memories of the subject are unclear, the notification means 96 notifies the family member terminal (step S206).

The dementia detection device 90 according to the second example embodiment can provide an opportunity for early detection of dementia by having a conversation with the subject using the AI model and notifying the family member of the subject based on an analysis result of the conversation.

Some or all of the above example embodiments (including the modification example, the same applies hereinafter) may also be described as the following supplementary notes, but are not limited to the following supplementary notes.

### Supplementary Note 1

A dementia detection device including:
a communication means for communicating with a subject terminal used by a subject and a family member terminal used by each of members of a family;
a storage means for storing personal information regarding the subject and each member and photograph information related to the subject based on consent of the subject and each member;
a photograph display means for transmitting the photograph information to the subject terminal and displaying the photograph information on the subject terminal;
a conversation means for having a conversation with the subject using an AI model based on the personal information and the photograph information via the subject terminal;
a memory determination means for determining whether memories of the subject are unclear by analyzing content of the conversation based on the personal information and the photograph information; and
a notification means for notifying the family member terminal in a case where the memories of the subject are unclear.

### Supplementary Note 2

The dementia detection device according to supplementary note 1, in which
the photograph information includes image data of a photograph and attribute data indicating memories of the subject related to the photograph,
the photograph display means transmits the image data of the photograph to the subject terminal and displays the image data on the subject terminal, and
the memory determination means determines whether the memories of the subject are unclear by comparing the content of the conversation with the subject related to the photograph with the attribute data of the photograph.

### Supplementary Note 3

The dementia detection device according to supplementary note 1, in which the notification means creates disclosed information to be disclosed to a medical institution for the subject, and notifies the family member terminal of a two-dimensional code indicating the disclosed information.

### Supplementary Note 4

The dementia detection device according to supplementary note 3, in which
the memory determination means creates evaluation data obtained by evaluating the memories of the subject in most recent and previous cases based on most recent conversation content related to the photograph and previous conversation content related to the photograph in a case where the memories of the subject are unclear,
the personal information includes medical claim data of the subject, and
the disclosed information includes the medical claim data and the evaluation data.

### Supplementary Note 5

The dementia detection device according to supplementary note 4, in which the evaluation data is a table obtained by evaluating three types of memories: memories regarding a time of the photograph, memories regarding a family shown in the photograph, and memories regarding a capture location of the photograph.

### Supplementary Note 6

The dementia detection device according to supplementary note 2, in which
the personal information includes facial image data of the subject and each member and family structure data indicating a family structure,
the dementia detection device further includes a photograph correction means for identifying a person shown in the photograph based on the image data of the photograph and the facial image data and linking the person with the photograph, and
the conversation means has a conversation with the subject based on the attribute data of the photograph, the member shown in the photograph, and the family structure data.

### Supplementary Note 7

The dementia detection device according to supplementary note 6, further including a photograph selection means for selecting a photograph corresponding to the content of the conversation by comparing the content of the conversation with the subject with the personal information and the photograph information,
in which the photograph display means transmits image data of the photograph corresponding to the content of the conversation to the subject terminal and displays the image data on the subject terminal.

### Supplementary Note 8

The dementia detection device according to supplementary note 7, in which
the photograph selection means identifies a member with whom the subject is having the conversation based on conversation image data showing the member with whom the subject is having the conversation and the facial image data, and selects a photograph showing the member, and
the photograph display means transmits image data of the photograph showing the member with whom the subject is having the conversation to the subject terminal and displays the image data on the subject terminal.

### Supplementary Note 9

A dementia detection method executed by a dementia detection device, the dementia detection method including:
communicating with a subject terminal used by a subject and a family member terminal used by each of members of a family;
storing personal information regarding the subject and each member and photograph information related to the subject based on consent of the subject and each member;
transmitting the photograph information to the subject terminal and displaying the photograph information on the subject terminal;
having a conversation with the subject using an AI model based on the personal information and the photograph information via the subject terminal;
determining whether memories of the subject are unclear by analyzing content of the conversation based on the personal information and the photograph information; and
notifying the family member terminal in a case where the subject memories are unclear.

### Supplementary Note 10

A program causing a computer to execute a process, the process including:
communicating with a subject terminal used by a subject and a family member terminal used by each of members of a family;
storing personal information regarding the subject and each member and photograph information related to the subject based on consent of the subject and each member;
transmitting the photograph information to the subject terminal and displaying the photograph information on the subject terminal;
having a conversation with the subject using an AI model based on the personal information and the photograph information via the subject terminal;
determining whether memories of the subject are unclear by analyzing content of the conversation based on the personal information and the photograph information; and
notifying the family member terminal in a case where the subject memories are unclear.

### Supplementary Note 11

The dementia detection device according to supplementary note 1, in which the photograph display means displays a photograph in which a face of a person other than those of a patient and a consenting member is blurred.

Although the present disclosure has been described with reference to the embodiments above, it is not limited to the aforementioned embodiments. Various modifications can be made to the structure and details of the present disclosure within the scope of the disclosure, as understood by those skilled in the art. That is, the present disclosure includes not only the embodiments as described but also various modifications and adaptations that would be apparent to those skilled in the art in accordance with the technical ideas underlying the disclosure, including the scope of the claims.

### DESCRIPTION OF SYMBOLS

1 Server
2 Subject terminal
3, 3a, 3b Family member terminal
11, 21 Interface
12, 22 Processor
13, 23 Memory
14, 24 Recording medium
15, 25 Display unit
16, 26 Input unit
31 Personal information DB
32 Photograph information DB
33 Conversation information DB
34 Disclosed information DB
51 Photograph correction unit
52 Photograph selection unit
53 Conversation content creation unit
54 Memory determination unit
65 Family contact unit
66 Disclosed information presentation unit
100 Dementia detection system

1. A dementia detection device comprising:
at least one memory configured to store instructions; and
at least one processor configured to execute the instructions to:
communicate with a subject terminal used by a subject and a family member terminal used by each of members of a family;
store personal information regarding the subject and each member and photograph information related to the subject based on consent of the subject and each member;
transmit the photograph information to the subject terminal and displaying the photograph information on the subject terminal;
have a conversation with the subject using an AI model based on the personal information and the photograph information via the subject terminal;
determine whether memories of the subject are unclear by analyzing content of the conversation based on the personal information and the photograph information; and
notify the family member terminal in a case where the memories of the subject are unclear.

2. The dementia detection device according to claim 1, wherein
the photograph information includes image data of a photograph and attribute data indicating memories of the subject related to the photograph,
the processor transmits the image data of the photograph to the subject terminal and displays the image data on the subject terminal, and
the processor determines whether the memories of the subject are unclear by comparing the content of the conversation with the subject related to the photograph with the attribute data of the photograph.

3. The dementia detection device according to claim 1, wherein the processor creates disclosed information to be disclosed to a medical institution for the subject, and notifies the family member terminal of a two-dimensional code indicating the disclosed information.

4. The dementia detection device according to claim 3, wherein
the processor creates evaluation data obtained by evaluating the memories of the subject in most recent and previous cases based on most recent conversation content related to the photograph and previous conversation content related to the photograph in a case where the memories of the subject are unclear,
the personal information includes medical claim data of the subject, and
the disclosed information includes the medical claim data and the evaluation data.

5. The dementia detection device according to claim 4, wherein the evaluation data is a table obtained by evaluating three types of memories: memories regarding a time of the photograph, memories regarding a family shown in the photograph, and memories regarding a capture location of the photograph.

6. The dementia detection device according to claim 2, wherein
the personal information includes facial image data of the subject and each member and family structure data indicating a family structure,
the processor is further configured to identify a person shown in the photograph based on the image data of the photograph and the facial image data and linking the person with the photograph, and
the processor has a conversation with the subject based on the attribute data of the photograph, the member shown in the photograph, and the family structure data.

7. The dementia detection device according to claim 6, wherein the processor is further configured to set a photograph corresponding to the content of the conversation by comparing the content of the conversation with the subject with the personal information and the photograph information,
wherein the processor transmits image data of the photograph corresponding to the content of the conversation to the subject terminal and displays the image data on the subject terminal.

8. The dementia detection device according to claim 7, wherein
the processor identifies a member with whom the subject is having the conversation based on conversation image data showing the member with whom the subject is having the conversation and the facial image data, and selects a photograph showing the member, and
the processor transmits image data of the photograph showing the member with whom the subject is having the conversation to the subject terminal and displays the image data on the subject terminal.

9. A dementia detection method executed by a dementia detection device, the dementia detection method comprising:
communicating with a subject terminal used by a subject and a family member terminal used by each of members of a family;
storing personal information regarding the subject and each member and photograph information related to the subject based on consent of the subject and each member;
transmitting the photograph information to the subject terminal and displaying the photograph information on the subject terminal;
having a conversation with the subject using an AI model based on the personal information and the photograph information via the subject terminal;
determining whether memories of the subject are unclear by analyzing content of the conversation based on the personal information and the photograph information; and
notifying the family member terminal in a case where the subject memories are unclear.

10. A non-transitory computer-readable recording medium storing a program causing a computer to execute a process, the process comprising:
communicating with a subject terminal used by a subject and a family member terminal used by each of members of a family;
storing personal information regarding the subject and each member and photograph information related to the subject based on consent of the subject and each member;
transmitting the photograph information to the subject terminal and displaying the photograph information on the subject terminal;
having a conversation with the subject using an AI model based on the personal information and the photograph information via the subject terminal;
determining whether memories of the subject are unclear by analyzing content of the conversation based on the personal information and the photograph information; and
notifying the family member terminal in a case where the subject memories are unclear.
